(19) United States
(12) Patent Application Publication
(10) Pub. No.: US 2011/0138682 A1
(43) Pub. Date: Jun. 16, 2011

(54) ALGAL CULTURE PRODUCTION, HARVESTING , AND PROCESSING

(75) Inventors: Priscila B. Demaris, Lake Charles, LA (US); Ramanujam Ravikumar, Lake Charles, LA (US); Philippe Vandevivere, Chassiers (FR)

(73) Assignee: AQUATIC ENERGY LLC, Lake Charles, LA (US)

(21) Appl. No.: 12/864,399

(22) PCT Filed: Jan. 22, 2009
(86) PCT No.: PCT/US09/31681

§ 371 Date: Dec. 22, 2010

Related U.S. Application Data
(60) Provisional application No. 61/023,572, filed on Jan. 25, 2008.

(51) Int. Cl. C10L 1/18; C12N 1/12; C12P 1/00; C12P 7/64; A23L 1/48; A01G 1/00

(52) U.S. Cl. 44/385; 435/257.1; 435/41; 435/257.3; 435/257.5; 435/257.6; 435/134; 426/7; 47/1.4

(57) ABSTRACT

Materials and methods are provided for growing algae while maintaining culture selectivity. Algae that can be grown include, for example, green algae such as those of the genus Scenedesmus. Lipid obtained from the algae can be used to produce biofuels such as biodiesel or polyunsaturated fatty acids such as omega-3 fatty acids. Feedstocks such as animal feed and aquaculture feed can also be produced as can phytonutrients such as asataxanthin and beta-carotene.

This application claims priority to U.S. Provisional Patent Application 61/023,572 filed Jan. 25, 2008, and incorporates the same in its entirety.

### BACKGROUND OF THE INVENTION

Increasing global demand and environmental concerns have lead to a search for both alternative and greener sources of fuel, animal feed, pharmaceuticals, nutraceuticals, polyunsaturated fatty acids, phytonutrients, minerals, vitamins, and other products. One environmental source of these products is algae. Algae are a particularly attractive source as algae can be grown using land that could not normally be used for food production or other purposes. However, production of these products from algae presents several obstacles including selection of a suitable alga, developing suitable growth conditions for optimal lipid yield, and preventing contamination from undesired algal species and other organisms. These obstacles are multiplied when algal growth is pursued on a large scale in an outdoors setting where weather and contamination are a constant threat. Accordingly, there exists a strong need for new algal production technologies.

### SUMMARY OF THE INVENTION

The present invention provides a method of selectively cultivating a target alga. The method comprises growing the target alga in a first pond; diluting the target alga in the first pond; supplying a nutrient composition to the first pond; and maintaining culture selectivity in the first pond. This method and other methods of the invention can be used for production of lipids for biofuel such as biodiesel and for polyunsaturated fatty acids such as omega-3 fatty acids. This method and other methods of the invention can also be used for production of feedstocks such as animal feed and aquaculture feed. This method and other methods of the invention can be used for production of phytonutrients such as beta-carotene and astaxanthin.

The present invention provides a method of selectively cultivating a target alga of the genus Scenedesmus. The method comprises growing the target alga in a first pond; diluting the target alga in the first pond; supplying a nutrient composition to the first pond; and maintaining culture selectivity in the first pond.

The present invention provides a method of selectively cultivating the target alga Scenedesmus obliquus. The method comprises growing the target alga in a first pond; diluting the target alga in the first pond; supplying a nutrient composition to the first pond; and maintaining culture selectivity in the first pond.

The present invention provides a method of selectively cultivating the target alga Scenedesmus obliquus. The method comprises the following steps. The target alga is grown in a raceway pond. Carbon dioxide is added to the raceway pond if a pH of about 8.5 or higher is reached. A cooling liquid is added to the raceway pond if a temperature of 33° C. or higher is reached. The alga in the raceway pond is diluted by about 60% at about every 20 hours. A nutrient composition is supplied to the raceway pond at about the same time as the diluting step, wherein the nutrient composition comprises sodium bicarbonate, urea, trisodium phosphate, and ferrous chloride, with a sodium bicarbonate concentration of at least 2 mM and a nitrogen:phosphate ratio of at least about 15:1. A volume of the alga obtained during the diluting step is discharged into a stress pond that contains a deficit of nitrogen. The alga from the stress pond is harvested and dewatered. Lipid is extracted from the alga.

The present invention provides a biofuel, feedstock, polyunsaturated fatty acid, phytonutrient, and any other useful product produced by any method of the invention.

The present invention provides a selective open-air pond algal culture comprising a target alga. The target alga can be a green alga. The green alga can be of the genus Scenedesmus. The target alga can be a diatom. The pond can be a raceway pond.

### DETAILED DESCRIPTION

A method of selectively cultivating a target alga for lipid production is provided in accordance with the invention. This method and other methods of the invention can be used for production of lipids for biofuel such as biodiesel and for polyunsaturated fatty acids such as omega-3 fatty acids. This method and other methods of the invention can be used for production of feedstocks such as animal feed and aquaculture feed. This method and other methods of the invention can be used for production of phytonutrients such as beta-carotene and astaxanthin.

The target alga can be any suitable species of alga or one or more strain thereof. That is, while the target alga is generally a single species of alga, in some embodiments it can be a combination of two or more algal species and/or strains thereof. The target alga preferably comprises an alga that is capable of producing high levels of lipid under suitable conditions.

The target alga can comprise at least one green alga. In some embodiments, the target alga is a diatom. The target alga can be obtained, isolated, and domesticated from any source, natural or manmade. In some embodiments, the alga is obtained from a source local to the location of algal culture production. In some embodiments, the target alga is obtained from the state of Louisiana of the U.S. In some embodiments, the target algal is obtained in or near Lake Charles, La. The target alga can be a colonial alga. The isolation and purification of a target alga can be done by pipette, medium, light and temperature methods. In some embodiments, the isolated and purified strain of target alga can survive in lower temperatures such as less than 10° C. for a few days. Domestication of a target alga strain can include treating the strain in lower temperature, lesser light source and minimal nutrient media. The purified algal strain can be grown in 5 ml of medium and then scaled up to several thousands of liter of medium, natural water, or treated water. Axenic cultures can be prepared from clean water such as reverse osmosis (RO) or distilled water. The strain of target alga can then introduced to the filtered or non-filtered source water or treated water for acclimatization. Aliquots of axenic cultures can be maintained in clean water as stock culture.

In some embodiments, the target alga comprises one or more green alga of the genus Scenedesmus or any combination thereof. In some embodiments, the green alga comprises Scenedesmus obliquus. In some embodiments, the green alga is selected from the group consisting of Scenedesmus obliquus, Scenedesmus quadricauda, Scenedesmus maximus, Scenedesmus aramatus, Scenedesmus opoliensis, Scenedesmus dimorphus, and any combination thereof. Variants of the species can be used. For example, Scenedesmus quadricauda maximus can be employed. The Scenedesmus obliquus can, for example, comprise the Scenedesmus obliquus University of Texas (UTEX) strain 1450.

Non-Scenedesmus algae and other aquaculturable microbes can also be employed in accordance with the invention. In some embodiments, the target alga comprises one or more green alga of the genus Chlorella such as Chlorella minutissima or any combination thereof. In some embodiments, the target alga comprises one or more green alga of the genus Botryococcus such as Botryococcus braunii, Botryococcus sueditica, or any combination thereof. In some embodiments, the target alga comprises one or more green alga of the genus Chlamydomonas or any combination thereof. In some embodiments, the target alga comprises one or more green alga of the genus Closterium or any combination thereof. In some embodiments, the target alga comprises one or more green alga of the genus Pediastrum or any combination thereof. In some embodiments, the target alga comprises one or more green alga of the genus Melosira or any combination thereof. In some embodiments, the target alga comprises one or more green alga of the genus Oedogonium or any combination thereof. In some embodiments, the target alga comprises one or more green alga of the genus Haematococcus such as Haematococcus pluvialis or any combination thereof. In some embodiments, the target alga comprises one or more green alga of the genus Dunaliella such as Dunaliella salina, Dunealiella parva, Dunealiella viridis or any combination thereof. In some embodiments, the target alga comprises one or more Prymnesiophycean green alga of the genus Isochrysis such as Isochrysis galpana or any combination thereof. In some embodiments, the target alga comprises one or more Prasinophycean green alga of the genus Tetraselmis such as Tetraselmis suecica or any combination thereof. In some embodiments, the target alga includes one or more diatom. Examples of diatoms include, but are not limited to, those of the genus Skeletonema such as Skeletonema costatum, Chaetoceros such as Chaetoceros calcitrans, or any combination thereof. A method of the invention described herein with respect to one particular alga can also be used by substituting or adding other alga described herein or otherwise known.

In some embodiments, the target alga is produced from a substantially pure culture. In some embodiments, the target alga is selected from a population of algal cultures. The target alga in the first pond can be maintained for any suitable time in the first pond. The algal culture volume in the first pond can be achieved by ramping up a starter culture of the target alga to achieve growth of the target alga in the first pond. In some embodiments, the ramping step comprises two or more steps of successively greater volumes of target alga.

Culture selectivity in accordance with the present invention does not require a monoculture of the target alga. The maintenance of culture selectivity comprises maintaining the target alga as the predominant alga in the algal culture of the first pond. There can be a temporary loss of culture selectivity, for example, when ramping up the algal culture, or during or following weather or other events. In some embodiments, the target alga is maintained to be at least 50% of the total algae. In some embodiments, the target alga is maintained to be at least 75% of the total algae. In some embodiments, the target alga is maintained to be at least 90% of the total algae. In some embodiments, the target alga is maintained to be at least 95% of the total algae. In some embodiments, the target alga is maintained to be at least 99% of the total algae. The open pond culture can comprise a 100% pure strain of the target alga or can be at least 90% pure. In some embodiments, the open pond culture can be at least 50% pure. In some embodiments, other species of algae are grown with the target alga for research or general production purposes.

The method of selectively cultivating a target alga comprises growing the target alga in a first pond; diluting the target alga in the first pond; supplying a nutrient composition to the first pond; and maintaining culture selectivity in the first pond. In some embodiments, the supplying the nutrient composition step is performed at about the same time as the diluting step. In some embodiments, the pH of the culture is maintained at from about pH 6 to about pH 8. The method can further comprise the step of adding carbon dioxide to the first pond if a pH of about 8.5 or higher is reached. The addition of carbon dioxide to maintain pH can be carried out in conjunction with or independent from the use of carbon dioxide as a nutrient source.

The method can further comprise the step of adding a cooling liquid to the first pond if a temperature of 33° C. or higher is reached. In some embodiments, the cooling liquid comprises fresh medium. When “medium” is referred to any suitable medium or media can be employed unless otherwise specified. For example, a medium with 5 mM sodium bicarbonate, 1 mM urea (or sodium nitrate or ammonia), 30 μM trisodium phosphate, and 2 μM ferrous chloride can be used. In some embodiments, reverse osmosis water is used to make the medium.

Any suitable nutrient composition can be employed with the invention. In some embodiments, the nutrient composition comprises sodium bicarbonate at a concentration of at least about 0.6 mM as measured after addition of the nutrient composition to the pond. In some embodiments, the nutrient composition comprises sodium bicarbonate at a concentration of at least about 2 mM as measured after addition of the nutrient composition to the pond. The nutrient composition can comprise a source of iron. In some embodiments, the source of iron comprises ferrous chloride. The nutrient composition can comprise a nitrogen source and a phosphate source. In some embodiments, the nitrogen source comprises urea and the phosphate source comprises trisodium phosphate. In some embodiments, the ratio of nitrogen to phosphate is at least about 15:1. In some embodiments, the ratio is at least about 29:1. In some embodiments, the ratio is about 30:1.

Any suitable structure or combination of structures can be used for the first pond. The first pond can be a raceway pond. A raceway pond provides a housing that allows the target alga in culture to move in a circuit. Any suitable circuit geometry can be employed. For example, the shape of the raceway pond can approximate that of a racing or running track. The pond can comprise parallel rectangular channels with semi-circular or sufficiently curved channels on either end joining neighboring ends of the parallel rectangular channels to form a continuous channel. The raceway pond can comprise one or more lanes of equal or differing dimensions. In some embodiments, the pond is divided evenly into two lanes with the width of each lane staying constant throughout the course of the pond.

The first pond can comprise a transparent housing. The housing can be completely or partially transparent. In some embodiments, the transparent housing comprises an acrylic polymer. However, any suitable material allowing the passage of light can be used for transparent housing.

The size of the first pond can be any suitable size. The volume (capacity) of the pond is provided so as to accommodate at least the algal culture volume. The volume of the pond can comprise further volume so as to allow for precipitation and other liquid entry to minimize or eliminate overflow. For example, a pond with a 22 liter capacity can suitably accommodate an algal culture volume of about 18 liters. In some embodiments, the algal culture volume of the first pond is about 18 liters or more. In some embodiments, the algal culture volume of the first pond is about 600 liters or more. In some embodiments, the algal culture volume of the first pond is about 14,000 liters or more.

The depth of the algal culture in the first pond is any suitable depth. The depth can be provided such that the amount of algae is balanced by the algae's access to sunlight. In some embodiments, the first pond comprises an average algal culture depth of about 13 to 20 centimeters. In some embodiments, the average algal culture depth is about 18 centimeters.

The target alga in the first pond can be mixed at any suitable speed. A suitable speed can be one that provides access of algal cells to sunlight and nutrients. In some embodiments, the target alga is mixed at a speed of about 12 cm/sec, about 15 cm/sec, or about 18 cm/sec. The mixing can be provided by any suitable means. In some embodiments, the mixing is provided using one or more paddlewheels. Fresh culture and medium can be added just prior to the paddlewheel. In some embodiments, the paddlewheel has at least six paddles and supports between the ends of each paddle. The paddlewheel can be positioned so that it straddles the median divide and outside wall of the pond. In some embodiments, the paddlewheel is placed so that it is able to push the culture the greatest distance before the lane curves. The number of paddlewheels employed can depend on the width of the pond. In some embodiments, there are between 1 and 3 paddlewheels employed. If more than one paddle wheel is used, they can be placed in parallel. The number and positioning of the paddlewheels can vary with the material used to make the paddlewheels and the strength thereof.

The target algal in the first pond can be diluted to any suitable degree and at any suitable frequency. The dilution can be continuous, substantially continuous, or staggered. In some embodiments, a relatively large volume of algal culture is removed relatively infrequently. In some embodiments, a relatively small volume of algal culture is removed relatively frequently. The target alga can be diluted by any suitable means. Medium can be added so as to dilute the algal culture, algal cells can be removed, or dilution can occur by a combination thereof. The removal of algal culture and addition of medium need not be simultaneous. The target alga can be diluted in any suitable quantity so as to maintain a substantially steady growth of algae in the first pond as well as utilizing the algae of the first pond for other uses. In some embodiments, the growth of algae is logarithmic for at least a portion of the time spent in the first pond. In some embodiments, the diluting step comprises diluting the target alga in the first pond by a dilution of from about 35% to about 60%. In some embodiments, the dilution is about 50%. In some embodiments, the dilution is performed about every 20 hours. Algal concentration can be measured using any suitable method. In some embodiments, the dilution is performed when a Secchi (black and white) disc reading of 5-6 cm is attained (when the disc is no longer visible). In some embodiments, the concentration of algae is maintained in a range of from about 2 million to about 3 million algae per ml in the first pond. The volume of algal culture removed from the first pond can depend on the percent dilution and the volume of the culture. This volume can be more than about 20% and less than about 60% of the total culture volume of the first pond. The algal concentrations of the removed volume can depend on whether the dilution is continuous or staggered. Cell counts can range from about 2.5 million cells/ml to about 5 or about 6 million cells/ml. The dilution amount and frequency can be adjusted to account for differences in sunlight. For example, adjustment can be made based on the time of year, season, hemisphere, and/or latitude. The particular species and strain of target alga can also be varied by such parameters. For example, one strain can be used during a winter or cold season, and another during a summer or warm season.

The diluting step can comprise removing a volume of the target alga from the first pond. In some embodiments, the volume of the target alga from the first pond is discharged into a second pond. In some embodiments, the removal of the volume of the target alga from the first pond and its discharge into a second pond is substantially simultaneous. In other embodiments, the removal from the first pond and the discharge into the second pond are separated by a suitable period of time.

The algal depth of the second pond can be any suitable depth. In some embodiments, the algal depth of the second pond is about 18 centimeters to about 30 centimeters. The retention time of the target alga once discharged into the second pond can be for any suitable time period. In some embodiments, the retention time is about 3 days. The second pond provides sufficient capacity to hold the volume of algal culture discharged into the second pond over the retention period. For example, when the retention time is about three days, and algal culture is added to second pond each day, the pond should hold 3 days of “flowed” (added) culture, with the volume being three times each flowed culture. In some embodiments, the concentration in the second pond ranges from 5 to 10 million cells per ml.

The second pond can comprise any suitable structure or combination of structures. Any suitable set of conditions can be maintained in the second pond. The second pond can be a stress pond. The second pond can be a settling pond. In some embodiments, the second pond is both a stress pond and a settling pond. A stress pond provides an environment that causes the target alga to increase production of lipids that can be harvested for biofuel production. The stress pond environment can be achieved in a number of different ways. For example, the target alga can be starved of nutrients generally or be deprived of one or more nutrient. In some embodiments, the stress pond is nitrogen deficient. Nitrogen deficiency can be complete or partial. Other nutrients, besides nitrogen, including carbon dioxide can be added to the algal culture in the second pond. The conditions in the second pond can be provided so as to maximize lipid production or other desired product by the target alga. In some embodiments, culture selectivity is maintained in the second pond. In some embodiments, the second pond is a stress pond and is similar to the design of the first pond, for example, a raceway pond, except deeper. A settling pond allows the target alga to settle. In some embodiments, the settling pond is funnel-shaped. In embodiments where the stress pond and settling pond are not the same pond, the second pond can be the stress pond, and a third pond is employed as the settling pond.

The target alga can be harvested from the second pond for use in downstream processes such as lipid extraction and ultimately biofuel production. The target alga can be harvested using any suitable means, in any suitable amount, and at any suitable frequency. In some embodiments, the harvesting is performed at a time about 52 hours to about 54 hours following discharge of the volume of the target alga into the second pond. In some embodiments, the harvesting is performed about 72 hours following discharge of the volume of the target alga into the second pond. In some embodiments, the harvesting is performed once a lipid concentration of at least about 25% of the cell mass is reached. Lipid content can be determined using any suitable measurement. In some embodiments, lipid content is measured using a fluorometer. The target reading for the fluorometer can depend on the chosen aperture and concentration of the sample. Any suitable fluorescent dye can be employed. Examples include Nile red, Nile blue and India blue.

The target alga can be dewatered. Dewatering can be performed as part of the harvesting step or as a separate step. In some embodiments, the dewatering step comprises employing at least one of a beltpress and a dehydrogenator. In some embodiments, the harvesting step comprises dewatering of the target alga achieved by pumping of settled target alga from the second pond. Aluminum sulfate (50-100 ppm for example), or ferric chloride (10-30 ppm for example) can be used to help the algae settle. A polymer (0.5% of algal biomass for example) can be used to facilitate coagulation of the algae before using a belt press. Any suitable polymer or combination of polymers can be employed. In some embodiments, an emulsion polymer is used. Examples of emulsion polymers include Flopam EM 640, Flopam EM 840, and combinations thereof. In some embodiments, a solution polymer is employed. More solution polymer may be required than if an emulsion polymer is employed. In addition or in the alternative, coagulation facilitators can include one or more of a clay, pH adjustment (an increase in pH for example), nutrient deficiency, and charged electrodes.

In some embodiments, the algae in an open pond is harvested after reaching a density of 3 million cells per milliliter and above, and passed through a 30 micron or higher mesh size filters depending upon the filtration rate. This filtered product is algal paste that can be treated with solvents like methanol, chloroform, acetone, ethanol, hexane etc, to extract lipid and purified to obtain bio diesel. Extraction of omega-3 fatty acids, animal feed such as aquaculture feed, beta-carotein, vitamins etc. can also extracted from various species of algae including micro algae. The pond water after filtering the algal mass can be treated through UV fluorescent exposure for 60 minutes or longer. In some embodiments, duration is extended up to three hours or more. The UV treated water can be pumped back into a pond and supplied with various nutrients such as nitrogen, phosphate and carbon dioxide. Fresh inoculum can be pumped in to the pond for algal growth. In some embodiments, nutrients of carbon, nitrogen, phosphorous, minerals, vitamins are added. In some embodiments, major nutrients are alone added to the culture pond.

Lipid can be extracted from the target alga. Any suitable means of lipid extraction can be employed. In some embodiments, the extracting step comprises at least one of chloroform:methanol extraction and hexane extraction. The algal mass can be treated with solvents such as a procedure of Bligh and Dyer, Fajardo and a supercritical CO₂ process to extract the lipids. The lipids may be processed to biodiesel using, e.g., transesterification process with alkali described by Holup and Skeaff. Bio-ethanol, bio-hydrogen, bio-methanol, and other products can be generated in addition or in the alternative.

The byproducts of biodiesel production processes such as omega 3 fatty acids and other groups of polyunsaturated fatty acids (PUFAs) are extracted from the algal paste. Even if biodiesel is not produced, these desirable lipids can be obtained and so need not be considered byproducts. Major omega-3 fatty acids include alpha-linolenic (ALA), docosahexaenoic acid (DHA) and eicosapentaenoic (EPA). The omega-3 fatty acids and PUFAs can be used in pharmaceutical and nutraceutical applications. The omega-3 fatty acids can be obtained as a by product during the lipid extraction process by treating the lipids under different temperature processes. In some embodiments, all these reactions are carried out in an anaerobic environment. In some embodiments a strain of target alga yields around greater than 22% of omega 3, greater than 29% of PUFAs, greater than 20% of monounsaturated fat and greater than 27% of saturated fat. In some embodiments, the algal lipid products can include approximately 26.1% omega C 18-3 fat, 20% monounsaturated fat, 26.4% polyunsaturated fat, 25% saturated fat, and 2.5% trans fat. Carbon chains can include, but are not limited to, C12 to C24 chains in different percentages. Actual lipid profiles can vary with increase or decrease of one or more components depending upon algal growth conditions. Other methods can also be employed. Omega-3 fatty acids can be used for various health applications such as prevention or treatment of medical disorders in the heart and circulatory system generally, inflammatory disorders, and cancer. Algae also have vitamin resources including: A, C, E that can be obtained using a vitamin extraction process from micro-algae.

The production of an algal meal feedstock can include the following steps. The algal paste obtained after extraction is treated with washed with anti-solvent, washed with deionized water, air dried and pasteurized at approximately 60° C. for around 12 hours. The biomass can then be milled and packed in appropriate containers as requested by a supplier. In some embodiments, algal meal products comprise 3% crude fiber, 0.1% calcium, 39% protein, 0.2% monounsaturated, 0.2% omega 3 fats, 0.2% polyunsaturated fats, 0.2% saturated fats, 0.1% trans fats, and 1% other fat. This biomass can also be used for the production of ethanol. Bio-gas can be produced from the anaerobic digestion of the biomass. Feedstocks of the invention can contain varying amounts of proteins, lipids, carbohydrates, fiber, minerals, vitamins, and other nutrients. The methods of the invention can be adjusted to produce such varying amounts.

The lipid content can be equal to or greater than 10%, 20%, 25%, 30%, 35%, 40%, or 50% of the algal paste. In some embodiments, the lipid content is 26.3% of the algal paste. The feedstock (meal) can be equal to or greater than 10%, 20%, 25%, 40%, 50%, 60%, 70%, 75%, 80%, 85%, 90%, or 95% of the algal paste. The protein content can be equal or greater than 10%, 20%, 25%, 30%, 35%, 40%, 45%, or 50% of the feedstock (meal). In some embodiments, the protein content is 39% protein.

A method of selectively cultivating a target alga of the genus Scenedesmus for is provided in accordance with the invention. The method comprises growing the target alga in a first pond; diluting the target alga in the first pond; supplying a nutrient composition to the first pond; and maintaining culture selectivity in the first pond.

The present invention provides a method of cultivating the target alga Scenedesmus obliquus. The method comprises growing the target alga in a first pond; diluting the target alga in the first pond; supplying a nutrient composition to the first pond; and maintaining culture selectivity in the first pond.

The present invention provides a method of selectively cultivating the target alga Scenedesmus obliquus. The method comprises the following steps. The target alga is grown in a raceway pond. Carbon dioxide is added to the raceway pond if a pH of about 8.5 or higher is reached. A cooling liquid is added to the raceway pond if a temperature of 33° C. or higher is reached. The target alga in the raceway pond is diluted by about 60% at about every 20 hours. A nutrient composition is supplied to the raceway pond at about the same time as the diluting step, wherein the nutrient composition comprises sodium bicarbonate, urea, trisodium phosphate, and ferrous chloride, with a sodium bicarbonate concentration of at least 2 mM and a nitrogen:phosphate ratio of at least about 15:1. A volume of the target alga obtained during the diluting step is discharged into a stress pond that contains a deficit of nitrogen. The target alga from the stress pond is harvested and dewatered. Lipid is extracted from the target alga.

Any method of the invention can further include the step of generating a biofuel from lipid produced from the target alga. Any suitable method can be employed. For example, transesterfication can be employed. In some embodiments, the biofuel is biodiesel. In some embodiments, the biofuel is bio-jet. The biofuel produced by any method of the invention is also an aspect of the invention. The present invention provides a biofuel produced by any method of the invention.

Any method of the invention can further include the step of generating a polyunsaturated fatty acid from the target alga. In some embodiments, the polyunsaturated fatty acid includes an omega-3 fatty acid. In some embodiments, the omega-3 fatty acid includes alpha-linolenic (ALA), docosahexaenoic acid (DHA), eicosapentaenoic (EPA), or any combination thereof. The present invention provides a polyunsaturated acid produced by any method of the invention.

Any method of the invention can further include the step of generating a feedstock from the target alga. The feedstock can be animal feed, aquaculture feed, or any combination thereof. The present invention provides a feedstock produced by any method of the invention.

Any method of the invention can further include the step of generating a phytonutrient from the target alga. The phytonutrient can be a carotenoid. In some embodiments, the carotenoid is astaxanthin, beta-carotene, or any combination thereof. The present invention provides a phytonutrient produced by any method of the invention.

A selective open-air pond algal culture comprising a target alga of the genus Scenedesmus is provided in accordance with the invention. The culture can be in a pond. The pond can be a raceway pond. As described above in respect to the methods of the invention, the selective algal culture need not be a monoculture. In some embodiments, the target alga is at least 50% of the total algae. In some embodiments, the target alga is at least 75% of the total algae. In some embodiments, the target alga is at least 90% of the total algae. In some embodiments, the target alga is at least 95% of the total algae. In some embodiments, the target alga is at least 99% of the total algae. The selective open-air pond algal culture can comprise Scenedesmus obliquus. The target alga can be selected from the group consisting of Scenedesmus obliquus, Scenedesmus quadricauda, Scenedesmus maximus, Scenedesmus opoliensis, Scenedesmus aramatus, Scenedesmus dimorphus and any combination thereof. Variants of the species can be used. For example, Scenedesmus quadricauda maximus can be employed. In some embodiments, the Scenedesmus obliquus comprises Scenedesmus obliquus UTEX strain 1450.

A selective open-air pond algal culture comprising a non-Scenedesmus target alga and/or other aquaculturable microbes can also be employed in accordance with the invention. In some embodiments, the culture comprises one or more green alga of the genus Chlorella such as Chlorella minutissima or any combination thereof. In some embodiments, the culture comprises one or more green alga of the genus Botryococcus such as Botryococcus braunii, Botryococcus sueditica, or any combination thereof. In some embodiments, the culture comprises one or more green alga of the genus Chlamydomonas or any combination thereof. In some embodiments, the culture comprises one or more green alga of the genus Closterium or any combination thereof. In some embodiments, the culture comprises one or more green alga of the genus Pediastrum or any combination thereof. In some embodiments, the culture comprises one or more green alga of the genus Melosira or any combination thereof. In some embodiments, the culture comprises one or more green alga of the genus Oedogonium or any combination thereof. In some embodiments, the culture comprises one or more green alga of the genus Haematococcus such as Haematococcus pluvialis or any combination thereof. In some embodiments, the culture comprises one or more green alga of the genus Dunaliella such as Dunaliella salina, Dunealiella parva, Dunealiella viridis or any combination thereof. In some embodiments, the culture comprises one or more Prymnesiophycean green alga of the genus Isochrysis such as Isochrysis galpana or any combination thereof. In some embodiments, the culture comprises one or more Prasinophycean green alga of the genus Tetraselmis such as Tetraselmis suecica or any combination thereof. In some embodiments, a diatom is the target alga or used in combination with one or more green alga for the culture. Examples of diatoms include, but are not limited to, those of the genus Skeletonema such as Skeletonema costatum, Chaetoceros such as Chaetoceros calcitrans, or any combination thereof. The culture can be in a pond. The pond can be a raceway pond. In some embodiments, the target alga is at least 50% of the total algae. In some embodiments, the target alga is at least 75% of the total algae. In some embodiments, the target alga is at least 90% of the total algae. In some embodiments, the target alga is at least 95% of the total algae. In some embodiments, the target alga is at least 99% of the total algae.

The following examples further illustrate the invention but, of course, should not be construed as in any way limiting its scope.

### EXAMPLE 1

This example demonstrates the growth of a green algal culture while maintaining culture selectivity in accordance with the present invention. Scenedesmus obliquus culture (University of Texas) is employed. To increase volume, a slant (20 mL at 0.5 million cells/mL) is sub-cultured into 6 test tubes (50 mL of culture until a concentration of 1 million cells/mL reached), using a UTEX nutrient medium although other suitable media can be used. The UTEX nutrient medium is a proteose medium of Bristol medium containing 1 g/L of proteose peptone. Bristol medium is 2.94 mM NaNO₃, 0.17 mM CaCl₂.2H₂O, 0.3 mM MgSO₄.7H₂O, 0.43 mM K₂HPO₄, 1.29 mM KH₂PO₄, and 0.43 mM NaCl. Once growth has been established, the cultures are transferred to 250 ml Erlenmeyer flasks at which point nutrient concentrations begin, these concentrations are described below. When the cell density increases (to a concentration of 1 million cells/ml in 200 ml of culture), the cultures are transferred to 1.5 liter bubble columns (1.25 L culture grown until 2 million cells/ml), continuing the same nutrient treatments. The cultures are next transferred into outdoor raceway ponds (each pond having a capacity of about 22 liters holding about 18 liters of algal culture). Cells concentrations in the ponds are maintained at from 2 million to 3 million cells/ml. Acrylic ponds are employed to ensure adequate light with a mixing speed of about 15 cm/s.

The nutrient concentrations, employed as referenced above and to maintain the pond cultures comprise sodium bicarbonate, urea, trisodium phosphate, and ferrous chloride. The concentrations expressed are those obtained after addition of nutrients to the ponds. Sodium bicarbonate is used at a concentration of 2 mM. A nitrogen to phosphate ratio (N:P) of about 30:1 is used at 0.75 mM N and 20 μM P. Ferrous chloride is used at about 2 μM. S. obliquus grows well within a pH range of 6-8. To achieve that, carbon dioxide is bubbled periodically throughout the day as soon as the pH reaches 8.5.

Scenedesmus obliquus has a doubling rate of about 20 hours. By keeping the cell retention time to about 20 hours, the alga is able to maintain consistent growth while other organisms with longer retention times are flushed out. In order to achieve this retention time, the culture is diluted by 60% everyday.

The temperature range at which S. obliquus grows best is between 20° C. and 30° C. However, at 35° C. the growth declines sharply. To keep the temperature in the optimal range, the ponds are maintained at a minimum depth of 18 centimeters at a mixing speed of 15 cm/s. Temperatures are monitored hourly and when exceeding 33° C. the culture is diluted with fresh medium.

To increase lipid content, the excess biomass from the daily dilutions is transferred to a deeper stress pond, where the culture grows until substantially all of the nitrogen is depleted. Because the nutrient concentration provided in the raceway pond is enough nitrogen for 24 hours of growth, the stress culture is nitrogen depleted in about 4-6 hours. The culture then remains stressed of nitrogen for 48 hours before harvesting.

Lipid analysis is performed using both fluorescence and total lipid extraction. Fluorescence can be a method for lipid measurement. The dye Nile Red is highly fluorescent in the presence of lipids and used to achieve readings. A Turner model 110 fluorometer with a F4T5/d lamp is employed. Emission filters employed are 420-470 nm and excitation filters employed are >520 nm. For this procedure, the culture is diluted to a biomass of 3 ppm. The dye is then added at a concentration of 1 ppm. This solution is mixed using a vortex mixer for 5 minutes, and results are then read at 5 minute intervals for one hour. The results are compared against a standard solution of 1 ppm triolein with 1 ppm Nile Red.

Total lipid extraction is performed using a modified Bligh and Dyer method. Chloroform and methanol are used in a 1:1 ratio to extract lipids useful for biodiesel production. The target alga is first dewatered and the slurry is dried over night using a bench-top dehydration unit. The algae flakes are then weighed and an equal amount of the chloroform methanol solution is added. This slurry is then mixed using the vortex. After 30 minutes the test tube is uncapped and the solvents allowed to evaporate. Once the evaporation is done, the contents are filtered and measured.

The methods for harvesting Scenedesmus obliquus can vary. In order to create biodiesel, the algae slurry is dewatered, and not completely dried. An inexpensive and fairly efficient way to dewater is to use a settling pond that also serves as the stress pond. This dual-purpose pond allows the algae to accumulate lipids while providing a storage place for harvesting. During the growth phase S. obliquus maintains a negative charge around the cell wall. This charge causes the cells the repel each other. Once the cell becomes older and is not photosynthesizing as rapidly, it loses the charge and is able to aggregate with other cells. These clumps become large and eventually sink to the bottom of the pond, allowing the thicker slurry to be pumped out. The cells reach this stationary phase while in the stress pond. As the cells accumulate lipids, they also begin to clump and settle.

### EXAMPLE 2

This example demonstrates the growth of a target algal culture for production of beta-carotene in accordance with the present invention. Beta carotene is a lipid and oil soluble product, which has antioxidant, free radical trapping properties and cancer preventive activity. Various species of algae can be cultivated to obtain beta-carotene globules. For example, marine, and sometimes freshwater, algae of the genus Dunaliella can be employed such as D. salina, D. parva, D. viridis and any combination of the same in basal medium. Dunaliella are unicellular, biflagellated, naked green algae. D. parva and D. salina can accumulate large quantities of beta-carotene. These algae can be grown in the range of 20 to 40° C., but can also tolerate much lower temperatures.

The followed can be used to prepare medium for algal beta-carotene production: 2.14 M NaCl, 4.81 μM FeCl₃, 1.82 μM MnCl₂, 0.13 mM NaH₂PO₄, and 1.18 mM NaNO₃, seawater and other minerals can also be employed. Productivities of 30-40 gm dry weight/m2/day can be achieved. Harvesting is done by high pressure filtration device using diatomaceous earth as a filter source. Harvested biomass may also be dried and can be marketed for consumption. In some cases, the algal mass is centrifuged or filtered and applied with NaCl followed by several cycles of centrifugation. The cells can be osmotically broken but the beta-carotene remains associated with the membranes. The beta-carotene globules are released at this step from the membranes to the supernatant and are present as a suspension. The suspension is mixed with solution containing 50% sucrose and Tris HCl, and the preparation is centrifuged. The purified beta-carotene globules are collected from the top layer, while the Chlorophyll containing membranes are pelleted at the bottom.

### EXAMPLE 3

This example demonstrates the growth of a diatomic or green algal culture for aquaculture feed in accordance with the present invention. The diatoms, Skeletonema costatum, Chaetoceros calcitrans, the Prymnesiophycean Isochrysis galpana and Prasinophycean Tetraselmis suecica can be grown in open ponds to produce aquaculture feed. The stock cultures are maintained at constant illumination of 2000 lux, at temperature ranges from 22-24° C. The diatoms are grown in a sea water medium containing NaNo₃, NaH₂PO₄, Na2SIO₃, FeCl₃, and Na₂EDTA. For the green algae, the silicate solution is omitted. The stock cultures are maintained in the laboratory and the culture is inoculated in to the open ponds. The optimal temperature is 20 to 33° C. The algae are harvested using a filter of 20 micrometers and the biomass is air dried and supplied as feed for the juvenile shrimps, oysters and other fish larvae. Products include not only aquaculture feed but also protein an fiber generally.

### EXAMPLE 4

This example demonstrates the growth of a target algal culture to produce astaxanthin in accordance with the present invention. Haematococcus pluvialis is grown in the laboratory and tested for Astaxanthin content. Astaxanthin is a carotenoid pigment and is used for various pharmaceutical and nutraceutical purposes. The alga is originally a green biflagellated chlorophycean member, normally grown in freshwater habitats. Each cell has a single cup shaped chloroplast with many pyrenoids. When the cells are stressed by factors such as high light intensity, nutrient depletion, direct exposure to the sunlight, etc. they form cysts, appear red in color that allows them to survive for a long period. The cysts accumulate large quantities of the red pigment, astaxanthin, in their cells, and it can reach up to 4% of its dry weight. The lab cultured H. pluvialis is stressed by high temperature and nutrient scarcity. The cysts are allowed to settle by gravitational force and treated with super critical CO₂ to break their cells. The ruptured cells release the accumulated astaxanthin that are moderately dried at room temperature and packed.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-88. (canceled)

89. A method of selectively cultivating a target alga, the method comprising:
growing the target alga in a first pond;
diluting the target alga in the first pond;
supplying a nutrient composition to the first pond; and
maintaining culture selectivity in the first pond.

90. The method of claim 89, further comprising adding carbon dioxide to the first pond if a pH of about 8.5 or higher is reached.

91. The method of claim 89, further comprising adding a cooling liquid to the first pond if a temperature of 33° C. or higher is reached.

92. The method of claim 89, wherein the step of diluting the target alga in the first pond comprises diluting the alga by about 35 to about 60% about every 20 hours.

93. The method of claim 89, wherein the diluting step comprises removing a volume of the target alga from the first pond.

94. The method of claim 89, further comprising supplying a nutrient composition to the first pond at about the same time as the diluting step.

95. The method of claim 94, wherein the nutrient composition comprises sodium bicarbonate, a nitrogen source, a phosphate source, and ferrous chloride, with a sodium bicarbonate concentration of at least 2 mM and a nitrogen:phosphate ratio of at least about 15:1.

96. The method of claim 95, wherein the nitrogen source comprises urea and the phosphate source comprises trisodium phosphate.

97. The method of claim 89, further comprising discharging a volume of the alga obtained during the diluting step into a second pond.

98. The method of claim 97, further comprising harvesting the alga from the second pond.

99. The method of claim 98, further comprising dewatering the alga harvested from the second pond.

100. The method of claim 98, further comprising extracting lipid from the alga.

101. The method of claim 98, further comprising preparing algal meal from the alga.

102. The method of claim 100, wherein the lipid extraction is performed by a method selected from the group consisting of chloroform:methanol extraction and hexane extraction.

103. The method of claim 102, wherein biodiesel is prepared from the extracted lipids by a method comprising transesterification with alkali.

104. The method of claim 101, wherein the algal meal is prepared by a method comprising extracting lipids from the alga to obtain an algal paste, washing the algal paste, and drying the algal paste.

105. The method of claim 104, further comprising pasteurizing and milling the algal meal.

106. The method of claim 89, wherein the alga is of a genus selected from the group consisting of Scenedesmus, Chlorella, Botryococcus, Chlamydomonas, Closterium, Pediastrum, Melosira, Oedogonium, Haematococcus, Dunaliella, Isochrysis, Tetraselmis, and any combination thereof.

107. The method of claim 106, wherein the alga is selected from the group consisting of Scenedesmus obliquus, Scenedesmus quadricauda, Scenedesmus maximus, Scenedesmus opoliensis, Scenedesmus aramatus, Scenedesmus dimorphus and any combination thereof.

108. The method of claim 100, wherein the alga is used to generate a product selected from the group consisting of biofuel, polyunsaturated fatty acid, feedstock, and phytonutrients.
